SEQUENCE LISTING (16 sequences)

SEQ ID NO: 1 - protein, 125 aa, synthetic construct
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDPNYDLWSGSLTKLDYWGQGTLVTVSS

SEQ ID NO: 2 - protein, 110 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSVVFGGGTKLTVL

SEQ ID NO: 3 - protein, 5 aa, synthetic construct
SYAMH

SEQ ID NO: 4 - protein, 17 aa, synthetic construct
VISYDGSNKYYADSVKG

SEQ ID NO: 5 - protein, 16 aa, synthetic construct
DPNYDLWSGSLTKLDY

SEQ ID NO: 6 - protein, 14 aa, synthetic construct
TGSSSNIGAGYDVH

SEQ ID NO: 7 - protein, 7 aa, synthetic construct
GNSNRPS

SEQ ID NO: 8 - protein, 10 aa, synthetic construct
QSYDSSLSVV

SEQ ID NO: 9 - DNA, 375 bp, synthetic construct
  source 1..375: mol_type = other DNA
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagagaccctaattacgacctttggagtggttcgctaacgaaacttgactactggggccagggaaccctggtcaccgtgtcctca

SEQ ID NO: 10 - DNA, 330 bp, synthetic construct
  source 1..330: mol_type = other DNA
cagtctgtgttgacgcagccgccctcagtgtctggggccccagggcagagggtcaccatctcctgcactgggagcagctccaacatcggggcaggttatgatgtacactggtaccagcagcttccaggaacagcccccaaactcctcatctatggtaacagcaatcggccctcaggggtccctgaccgattctctggctccaagtctggcacctcagcctccctggccatcactgggctccaggctgaggatgaggctgattattactgccagtcctatgacagcagcctgagtgtggtattcggcggagggaccaagctgaccgtccta

SEQ ID NO: 11 - DNA, 15 bp, synthetic construct
  source 1..15: mol_type = other DNA
agctatgctatgcac

SEQ ID NO: 12 - DNA, 51 bp, synthetic construct
  source 1..51: mol_type = other DNA
gttatatcatatgatggaagtaataaatactacgcagactccgtgaagggc

SEQ ID NO: 13 - DNA, 48 bp, synthetic construct
  source 1..48: mol_type = other DNA
gaccctaattacgacctttggagtggttcgctaacgaaacttgactac

SEQ ID NO: 14 - DNA, 42 bp, synthetic construct
  source 1..42: mol_type = other DNA
actgggagcagctccaacatcggggcaggttatgatgtacac

SEQ ID NO: 15 - DNA, 21 bp, synthetic construct
  source 1..21: mol_type = other DNA
ggtaacagcaatcggccctca

SEQ ID NO: 16 - DNA, 30 bp, synthetic construct
  source 1..30: mol_type = other DNA
cagtcctatgacagcagcctgagtgtggta

(19) United States
(12) Patent Application Publication
(10) Pub. No.: US 2025/0263505 A1
(43) Pub. Date: Aug. 21, 2025

(54) ANTIBODY AGAINST KYNURENINE

(71) Applicant: SALION GMBH, Münsing (DE)

(72) Inventors: Dietmar ABENDROTH, Thalfingen (DE); Manfred STANGL, Sauerlach (DE)

(21) Appl. No.: 18/857,989

(22) PCT Filed: Apr. 19, 2023
(86) PCT No.: PCT/EP2023/060188

§ 371 Date: Oct. 18, 2024

(30) Foreign Application Priority Data
Apr. 20, 2022 (EP) 22169087.8

(51) Int. Cl. C07K 16/44; G01N 33/53; G01N 33/68

(52) U.S. Cl. CPC C07K 16/44; G01N 33/5308; G01N 33/6893; C07K 2317/565; C07K 2317/92; G01N 2800/7095

(57) ABSTRACT

A monoclonal antibody, or an antigen-binding fragment thereof, or a synthetic and biotechnological derivative thereof that specifically binds kynurenine (KYN) and acts as a detection marker for inflammation in an individual can be provided.

Also, a reliable method and test kit for the detection of inflammation by determining the level of kynurenine in a body fluid, preferably saliva can be provided. Using embodiments of the inventive monoclonal antibody, a tool to determine the level of KYN easily and quickly without the need of any trained person when using saliva as the sample can be provided. This is also beneficial in pediatric medicine and monitoring of various diseases.

### CROSS-REFERENCE TO RELATED APPLICATIONS

The present application is the U.S. national stage of International Patent Application No. PCT/EP2023/060188, filed on Apr. 19, 2023 and claiming priority to European Patent Application No. EP 22169087.8, filed on Apr. 20, 2022.

### STATEMENT REGARDING SEQUENCE LISTINGS

A sequence Listing using exXtensible Markup Language (XML) compliant with World Intellectual Property Organization (WIPO) Standard ST.26 is provided herewith and the entirety of this sequence listing is incorporated by reference herein. The Sequence Listing that is incorporated by reference herein was e-filed at the USPTO using Patent Center as an XML file named SAL-P004WO.xml (16 KB in size) and was created on Apr. 19, 2023. This sequence listing was previously submitted with International Patent Application No. PCT/EP2023/060188.

### FIELD

The present invention relates to an antibody against kynurenine and methods of using the same.

### BACKGROUND

Kynurenine, KYN, is an aromatic, non-proteinogenic amino acid, and a metabolite of the tryptophan, TRP, metabolism. TRP is an essential amino acid that is not only required for the synthesis of proteins, but also for the biosynthesis of neurotransmitters like serotonin and melatonin. Most of the dietary TRP is catabolized via the kynurenine pathway, KP, to kynurenines by the enzymes tryptophan-2,3-dioxygenase, TDO, which is produced in the liver, and indoleamine-2,3-dioxygenase, IDO-1, which is produced in many tissues in response to immune activation. Kynurenines are involved in several physiological processes, including neurotransmission and immune responses. The KP involves neurotoxic and neuroprotective metabolites, and alterations in their delicate balance have been demonstrated in multiple pathological processes.

### SUMMARY

During an inflammation, which is the first line of defense of the innate immune system against dangerous proteins, the enzymes IDO-1 and TDO are activated by inflammatory cytokines like IFN-α, IFN-β, IFN-γ, TNFα and IL-6. Thus, the level of KYN is increased upon activation of the innate immune system.

An altered kynurenine pathway is associated with many diseases such as viral and bacterial infections, cancer, and diabetes. Imbalances in the kynurenine pathway have also been shown to be relevant in different disorders in which there is a cognitive decline. Changes in kynurenine metabolites have additionally been suggested to correlate with the infarct volume, the mortality of stroke patients and the post-stroke cognitive impairment.

Due to the involvement of KYN in various disease states, there is a need in the art to be able to determine the level of KYN quickly and easily. It would additionally be preferable if the measurement could be done by the individual itself without the requirement of a physician or any other trained person.

The method to determine the level of KYN preferably used today is HPLC (high performance liquid chromatography). This is a method that uses adsorption and flow rates to separate, identify, and quantify different components in a mixture. Another way used in the prior art is to determine the level of IDO-1 which is then extrapolated to the level of KYN. There are also antibodies described that bind to different components of the kynurenine pathway for use in Enzyme Linked Immunosorbent Assay (ELISA), immunofluorescence, immunochemistry and Western blotting of serum, plasma and/or dry blood samples.

The methods described in the prior art are not suitable to determine the amount of kynurenine in a sample quickly and easily, advantageously without the involvement of a physician or any other trained person.

Thus, there is a need for an antibody against KYN for the detection of said amino acid not only in blood samples but preferably also in other body samples like saliva that could be taken non-invasively by the patient himself/herself. This is especially significant in pediatric medicine and monitoring of various diseases.

Therefore, embodiments can be configured to provide an antibody that specifically binds to kynurenine. The antibody provided by the present invention is preferably capable to detect KYN in body fluids, preferably saliva, in which the level of KYN is low, and is preferably used in assays which can be performed by an individual itself.

A monoclonal antibody, or an antigen-binding fragment thereof, or a synthetic and biotechnological derivative thereof can be provided that specifically binds kynurenine and acts as a detection marker for inflammation in an individual.

According to a preferred embodiment, a monoclonal antibody, or an antigen-binding fragment thereof, or a synthetic and biotechnological derivative thereof is provided that binds kynurenine in a body fluid.

According to another preferred embodiment, the body fluid is serum, saliva and/or cerebro-spinal fluid (CSF). In a particularly preferred embodiment, the body fluid is saliva.

According to another preferred embodiment, the monoclonal antibody, or antigen-binding fragment thereof, or synthetic and biotechnological derivative thereof, is defined by one or more complementarity-determining regions (CDRs) and/or by variable heavy chain (VH) and optionally variable light chain (VL) regions comprising said CDRs or related sequences.

Further, according to a preferred embodiment, the monoclonal antibody, or antigen-binding fragment thereof, or synthetic and biotechnological derivative thereof comprises a variable region of the heavy chain (VH) which has the sequence as set forth in SEQ ID No: 1.

According to another preferred embodiment, the monoclonal antibody, or antigen-binding fragment thereof, or synthetic and biotechnological derivative thereof comprises a variable region of the light chain (VL) which has the sequence as set forth in SEQ ID No: 2.

According to yet another preferred embodiment, the monoclonal antibody, or antigen-binding fragment thereof, or synthetic and biotechnological derivative thereof comprises one or more complementarity-determining regions, CDRs, and/or a variable heavy chain (VH) region comprising an amino acid sequence having at least 90% sequence identity to the amino acid sequence of SEQ ID No: 1; and a variable light chain (VL) region comprising an amino acid sequence having at least 90% sequence identity to the amino acid sequence of SEQ ID No: 2.

According to yet another preferred embodiment, the monoclonal antibody, or an antigen-binding fragment thereof, or a synthetic and biotechnological derivative thereof is characterized by a VH region and optionally a VL region each comprising three CDRs designated as CDRH1, CDRH2, CDRH3, and CDRL1, CDRL2 and CDRL3, respectively, defining the binding specificity of the antibody, or antigen-binding fragment thereof, or synthetic and biotechnological derivative thereof.

According to some embodiments of the present invention, a VH region or the CDRs thereof alone may constitute a complete antigen-binding site. In certain embodiments, the antibody comprises a VH region or the CDRs thereof as defined herein alone. In certain embodiments, the antibody comprises a VH region or the CDRs thereof as defined herein together with a VL region or the CDRs thereof, particularly with a VL region or the CDRs thereof as defined herein.

Another preferred embodiment of the present invention relates to a monoclonal antibody, or antigen-binding fragment thereof, or synthetic and biotechnological derivative thereof, wherein at least one CDR is selected from:
heavy chain CDRs as defined by SEQ ID No: 3; SEQ ID No: 4; and SEQ ID No: 5; and
light chain CDRs as defined by SEQ ID No: 6; SEQ ID No: 7; and SEQ ID No: 8.

Further, according to a preferred embodiment, the monoclonal antibody, or antigen-binding fragment thereof, or synthetic and biotechnological derivative thereof comprises:
a heavy chain CDR1 (CDRH1) domain with an amino acid sequence as set forth in SEQ ID No: 3,
a heavy chain CDR2 (CDRH2) domain with an amino acid sequence as set forth in SEQ ID No: 4,
a heavy chain CDR3 (CDRH3) domain with an amino acid sequence as set forth in SEQ ID No: 5,
and
a light chain CDR1 (CDRL1) domain with an amino acid sequence as set forth in SEQ ID No: 6,
a light chain CDR2 (CDRL2) domain with an amino acid sequence as set forth in SEQ ID No: 7,
a light chain CDR3 (CDRL3) domain with an amino acid sequence as set forth in SEQ ID No: 8.

According to yet another preferred embodiment, the monoclonal antibody, or antigen-binding fragment thereof, or synthetic and biotechnological derivative thereof comprises a VH region comprising the CDRH1 of SEQ ID No: 3, the CDRH2 of SEQ ID No: 4, and the CDRH3 of SEQ ID No: 5, and optionally a VL region comprising the CDRL1 of SEQ ID No: 6, the CDRL2 of SEQ ID No: 7, and the CDRL3 of SEQ ID No: 8.

In another preferred embodiment, up to one, up to two, or up to three amino acid residues are substituted in CDRH1. In another preferred embodiment, up to one, up to two, or up to three amino acid residues are substituted in CDRH2. In yet another preferred embodiment, up to one, up to two, or up to three amino acid residues are substituted in CDRH3. In yet another preferred embodiment, up to one, up to two, or up to three amino acid residues are substituted in CDRL1. In still another preferred embodiment, up to one, up to two, or up to three amino acid residues are substituted in CDRL2. In yet another preferred embodiment, up to one, up to two, or up to three amino acid residues are substituted in CDRL3.

Further, in a preferred embodiment, up to one, up to two, or up to three amino acid residues are omitted in CDRH1. In another preferred embodiment, up to one, up to two, or up to three amino acid residues are omitted in CDRH2. In yet another preferred embodiment, up to one, up to two, or up to three amino acid residues are omitted in CDRH3. In another preferred embodiment, up to one, up to two, or up to three amino acid residues are omitted in CDRL1. In still another preferred embodiment, up to one, up to two, or up to three amino acid residues are omitted in CDRL2. In yet another preferred embodiment, up to one, up to two, or up to three amino acid residues are omitted in CDRL3.

According to another preferred embodiment, also in other parts of the antibody, or antigen-binding fragment thereof, or synthetic and biotechnological derivative thereof, amino acids can be exchanged by conservative amino acid substitution without changing the specificity of the antibody for KYN.

A preferred embodiment provides a monoclonal antibody, or an antigen-binding fragment thereof, or a synthetic and biotechnological derivative thereof. In a preferred embodiment, the isolated antigen-binding protein is a Fab fragment, a Fab′ fragment, a F(ab′)2 fragment, a Fv fragment, a diabody, a nanobody or a single chain antibody molecule. In another preferred embodiment, the isolated antigen-binding protein is of the IgG1-, IgG2-, IgG3- or IgG4-type. In yet another preferred embodiment, the antibody may be of the IgA-, IgD-, IgE- or IgM-type.

In some embodiments, a monoclonal antibody of the IgG1-Type can be provided. In yet another preferred embodiment, a scFv-fragment can be provided.

According to yet another preferred embodiment, the monoclonal antibody, or antigen-binding fragment thereof, or synthetic and biotechnological derivative thereof is capable to bind to kynurenine with an affinity (K_{D}) between 1×10⁻⁵ M and 1×10⁻⁸ M. In another preferred embodiment, the monoclonal antibody, or antigen-binding fragment thereof, or synthetic and biotechnological derivative thereof is capable to bind to kynurenine with an affinity (K_{D}) of less than 1×10⁻⁵ M, preferably less than 1×10⁻⁶ M, more preferably less than 1×10⁻⁷ M. In yet another preferred embodiment, the inventive antibody binds with a K_{D} of 1×10⁻⁵ M or less, preferably 1×10⁻⁶ M or less, more preferably 1×10⁻⁷ M or less.

According to still another preferred embodiment, the monoclonal antibody, or antigen-binding fragment thereof, or synthetic and biotechnological derivative thereof is human.

An isolated nucleic acid molecule can be is provided for encoding an embodiment of the inventive monoclonal antibody, or antigen-binding fragment thereof, or synthetic and biotechnological derivative thereof.

According to some embodiments of the present invention, an expression vector is provided comprising said nucleic acid molecule.

According to some embodiments of the invention, a method of producing the monoclonal antibody, or antigen-binding fragment thereof, or synthetic and biotechnological derivative thereof is provided. Embodiments of the method can include the steps of:
(i) introducing the expression vector mentioned above into an isolated host cell,
(ii) growing the cell under conditions permitting production of the antibody, or antigen-binding fragment thereof, or synthetic and biotechnological derivative thereof, and
(iii) recovering the antibody, or fragment, or derivative so produced.

According to some embodiments of the invention, an in vitro method for the detection of a disease or condition in an individual is provided, wherein the level of kynurenine in a body fluid is determined using the inventive monoclonal antibody, or an antigen-binding fragment thereof, or a synthetic and biotechnological derivative thereof, and wherein the value of kynurenine measured in the individual to be diagnosed is compared with the average value obtained from a comparable cohort of healthy persons, whereby the value of kynurenine in patients is increased.

According to another preferred embodiment, the determination of kynurenine in a body fluid is used for therapy control in a patient.

According to yet another preferred embodiment, the determination of KYN in a body fluid is used for monitoring of an individual's health status.

According to yet another preferred embodiment, the diagnostic use of the antibody or antigen-binding fragment thereof, or synthetic and biotechnological derivative thereof can be provided. Uses include, but are not limited to, the detection of inflammation, detection of various diseases and/or biological markers in a sample, preferably saliva.

According to still another preferred embodiment, the body fluid is serum, saliva or cerebro-spinal fluid (CSF). In a particularly preferred embodiment, the body fluid is saliva.

Further, according to a preferred embodiment, the level of kynurenine is at least two times higher in a patient than in the healthy control group. More preferably, the level of kynurenine in a patient is at least 3 μM in serum, at least 1 μM in saliva, and at least 1 μM in CSF. Even more preferably, the level of KYN in a patient is 4 μM to 25 μM, even more preferably 5 μM to 17 μM, still more preferably 5.5 μM to 12 μM, still more preferably 6 μM to 9 μM in serum. More preferably, the level of KYN in a patient is 1.5 μM to 4 μM in saliva. More preferably, the level of KYN in a patient is 1.5 μM to 4 μM in CSF.

In yet another preferred embodiment, the disease or condition to be detected is selected from at least one of neurodegenerative diseases, transplantation, infections, dialysis, dental diseases, sports medicine, gynecology, gastroenterology, cancer, diabetes, multiple sclerosis, asthma, and concussion.

More preferably, neurodegenerative diseases include, but are not limited to, Alzheimer's disease, Parkinson's disease, vascular dementia, Huntington's disease, and postoperative cognitive dysfunction.

More preferably, transplantation includes, but is not limited to, transplantation of solid organs, stem cell transplantation, cornea- and bone transplantation, graft vs. host disease.

More preferably, infections include, but are not limited to, infections with Severe acute respiratory syndrome Coronavirus type 2 (SARS-CoV-2), sepsis, tuberculosis, infections with Cytomegalovirus (CMV).

More preferably, sports medicine includes, but is not limited to, overtraining syndrome.

More preferably, gynecology includes, but is not limited to, eclampsia.

More preferably, gastroenterology includes, but is not limited to, Morbus Crohn.

More preferably, dental diseases include, but are not limited to, periodontitis.

Furthermore, embodiments of the inventive method can also be beneficial in monitoring the fitness of an individual.

According to another preferred embodiment, the in vitro method is an ELISA test, or a lateral flow immunochromatographic assay (LFA), or a microfluidic test.

Thus, embodiments of the in vitro method as disclosed herein may be based on different principles which will be explained in more detail in the description of the figures. One of the preferred principles is known as Lateral Flow Immunochromatographic Assay (LFA). Such a LFA test can be easily performed by the patient without the help of a physician or other professionals when saliva is used as the sample.

In another preferred embodiment, the single components of the lateral flow assay are adapted in such a manner that the presence of kynurenine is indicated only when more than a certain threshold value of kynurenine is present in the sample.

In another preferred embodiment, the in vitro method is performed as an ELISA. There are different configurations of ELISA tests known. ELISA types are direct ELISA, sandwich ELISA, competitive ELISA and/or reverse ELISA.

In still a further preferred embodiment, the in vitro test method is a microfluidic test. Said test can be used to conduct an ELISA or LFA test on paper. The test comprises two parts: a sliding strip which contains the active sensing area, and a structure surrounding the sliding strip, which holds stored reagents like buffers, antibodies, and enzymatic substrates, and distributes fluids. Running said test involves adding sample of a body fluid and water, moving the sliding strip at scheduled times, and analyzing the resulting color in the sensing area visually or using a flatbed scanner.

According to some embodiments of the invention, test kits with which the level of kynurenine in a body fluid is determined using the inventive antibody are provided. Such test kits comprise suitable means for performing the inventive method which may work on different principles. The kit comprises at least one antibody that specifically binds kynurenine.

In another preferred embodiment, the kit may comprise a second antibody. When two antibodies are used, such antibodies do not bind to the same epitope in order to allow the formation of a sandwich formed by a first antibody, which specifically binds kynurenine or its derivative, and a second antibody. Preferably, the second antibody is an enzyme-linked secondary antibody which is applied as a detection antibody.

According to a preferred embodiment, the test kit is an ELISA test kit, or a LFA test kit, or a microfluidic test kit.

According to still another preferred embodiment, the body fluid used in the test kit is serum, saliva, or CSF. In a particularly preferred embodiment, the body fluid is saliva.

A preferred test kit can consist of the following components:
1. Sample pad—an absorbent pad onto which the test sample is applied;
2. Conjugate or reagent pad—this contains antibodies specific to the target analyte conjugated to colored particles (usually colloidal gold particles, or latex microspheres);
3. Reaction membrane—typically a hydrophobic nitrocellulose or cellulose acetate membrane onto which anti-target analyte antibodies are immobilized in a line across the membrane as a capture zone or test line (a control zone may also be present, containing antibodies specific for the conjugate antibodies);
4. Wick or waste reservoir—a further absorbent pad designed to draw the sample across the reaction membrane by capillary action and collect it.

The components of the strip are usually fixed to an inert backing material and may be presented in a simple dipstick format or within a plastic casing with a sample port and reaction window showing the capture and control zones.

The term “antibody” as used herein, is intended to refer to any antigen binding molecule e.g. in some embodiments to immunoglobulin molecules comprised of four polypeptide chains, two heavy (H) chains and two light (L) chains interconnected by disulfide bonds. Each heavy chain is comprised of a heavy chain variable region (VH) and a heavy chain constant region (comprised of domains CH1, CH2, and CH3). Each light chain is comprised of a light chain variable region (VL) and a light chain constant region (CL).

The term “variable region” or “variable domain” as used herein refers to the domain of an antibody heavy or light chain that is involved in binding the antibody to the antigen. The variable regions of the heavy chain and light chain (VH and VL, respectively) of a native antibody generally have similar structures. They can be further subdivided into regions of hypervariability, termed complementarity-determining regions (CDRs), interspersed with regions that are more conserved, termed framework regions (FRs). The framework regions serve to position and align the CDRs in three-dimensional space. CDRs contain antigen-contacting residues and are primarily responsible for binding of the antigen. Generally, each VH and VL is composed of three CDRs and four FRs, arranged from amino-terminus to carboxy-terminus in the following order: FR1, CDR1, FR2, CDR2, FR3, CDR3, FR4. Unless otherwise indicated, CDR residues and other residues in the antibody of the present invention are numbered according to Kabat, et al. (Sequences of Proteins of Immunological Interest, 5th Ed., Public Health Service, National Institutes of Health, Bethesda, MD, 1991). The CDR amino acid sequences of the inventive antibody are set forth in SEQ ID Nos: 3 to 8, the nucleic acid sequences of the CDRs of the inventive antibody are set forth in SEQ ID Nos: 11 to 16. The amino acid sequence of the VH region of the antibody of the present invention is set forth in SEQ ID No: 1, the nucleic acid sequence of the VH region is set forth in SEQ ID No: 9. The amino acid sequence of the VL region of the antibody of the present invention is set forth in SEQ ID No: 2, the nucleic acid sequence of the VL region is set forth in SEQ ID No: 10. Silent mutations within the nucleic acid sequences, i.e. exchanges of bases that do not alter the amino acid sequence, are also encompassed according to the present invention and do not affect the properties of the antibody according to the invention.

Substitution of up to one, up to two, or up to three amino acid residues per CDR, of up to one, up to two, or up to three amino acid residues within all three CDRs of one light or heavy chain, or omission of one or more CDR(s) is also possible. Antibodies have been described in the scientific literature in which one or two CDR(s) can be dispensed with for binding. If a CDR or amino acid residue(s) thereof is/are omitted, it is/they are usually substituted with an amino acid occupying the corresponding position in another human antibody sequence or a consensus of such sequences. Positions for substitutions within CDRs and amino acids to substitute can also be selected empirically. Empirical substitutions can be conservative or non-conservative substitutions.

The term “monoclonal antibody” as used herein refers to an antibody obtained from a population of substantially homogeneous antibodies. In general, there are two types of antibodies, polyclonal and monoclonal. Polyclonal antibodies are mixtures of antibodies obtained from serum of immunized animals (usually goat, rabbit, mouse, or rat). By adsorption to an antigen in an affinity chromatography, the antibodies against the different epitopes of an antigen can be purified together, although the antibodies were produced by different B cells. Due to their way of production, polyclonal antibodies do show a wide range of specificity, and, consequently, are difficult to predict in their efficacy.

In contrast thereto, monoclonal antibodies are antibodies made by cloning a unique B cell. All subsequent antibodies derived this way trace back to a unique parent cell. Monoclonal antibodies can have a monospecific activity binding only to the same epitope (the part of an antigen that is recognized by the antibody). But also, bispecific monoclonal antibodies can be engineered by increasing the targets of one monoclonal antibody to two epitopes. Monoclonal antibodies show an advantageous binding profile compared to polyclonal antibodies. Further, the individual monoclonal antibodies comprising the population are identical and/or bind the same epitope, except for possible variant antibodies, e.g. containing naturally occurring mutations or arising during production of a monoclonal antibody preparation, such variants generally being present in minor amounts. In contrast to polyclonal antibody preparations, which typically include different antibodies directed against different epitopes, each monoclonal antibody of a monoclonal antibody preparation is directed against a single epitope on an antigen. Thus, the modifier “monoclonal” indicates the character of the antibody as being obtained from a substantially homogeneous population of antibodies and is not to be construed as requiring production of the antibody by any particular method.

The term “human antibody” as used herein, comprises antibodies having variable and constant regions derived from human germline immunoglobulin sequences. The human monoclonal antibodies of the invention may include amino acid residues not encoded by human germline immunoglobulin sequences (e.g., mutations introduced by random or site-specific mutagenesis in vitro or by somatic mutation in vivo), for example in the CDRs.

An “antibody fragment” as used herein, refers to a molecule other than a full length antibody that comprises a portion of a full length antibody that binds the same antigen to which the full length antibody binds. Examples of antibody fragments include, but are not limited to, Fv, Fab, Fab′, Fab′-SH, diabodies, linear antibodies, single-chain antibody molecules (e.g. scFv), and a fragment containing a CDR, or an isolated CDR. An antibody fragment of the present invention refers to one or more fragments of an antibody that retains the ability to specifically bind to kynurenine.

An “antigen-binding site” as used herein refers to a part of an antibody which recognizes and binds to an antigen. An antigen-binding site is formed by several individual amino acid residues from the antibody's heavy and light chain variable domains that are arranged in spatial proximity in the tertiary structure of the Fv region.

The term “scFv”, as used herein, refers to an antibody fragment format comprising variable regions of heavy (VH) and light (VL) chains, or two copies of a VH or VL chain of an antibody, which are joined together by a short flexible peptide linker which enables the scFv to form the desired structure for antigen binding. The scFv is a fusion protein of the variable regions of the heavy (VH) and light chains (VL) of immunoglobulins and can be easily expressed in functional form in E. coli or other host cells.

The term “synthetic and biotechnological derivative(s)”, as used herein, means any engineered fragment, synthesized by chemical or recombinant techniques, which possess(es) the desired antigen-binding specificity of the inventive antibody and retain(s) the functional properties of the antibody. For example, these derivatives can possess modifications in the region(s) which is/are not important for antigen-binding. Said antibody derivatives can for example be obtained from antibodies according to the present invention by the exchange of one or more constant region(s) and/or linkages with other molecules. Thus, an exchange of constant regions for an isotype can be carried out where, for example, an antibody of class IgM can be converted in an antibody of class IgG, with maintenance of its antigen specificity. This isotype switch can take place by cell biological or molecular biological methods which are well-known in the art. Another possibility of providing derivatives of the antibody according to the present invention is an exchange of amino acids in other parts of the antibody by conservative amino acid substitution.

A “conservative amino acid substitution” is one in which an amino acid residue is substituted by another amino acid residue having a side chain (R group) with similar chemical properties (e.g., similar charge or hydrophobicity). In general, a conservative amino acid substitution will not substantially change the functional properties of a protein. In cases where two or more amino acid sequences differ from each other by conservative substitutions, the percent of degree of similarity may be adjusted upwards to correct for the conservative nature of the substitution. Means for making this adjustment are well known to those of skill in the art.

Examples of groups of amino acids that have side chains with similar chemical properties, and whose substitution of each other constitutes conservative substitutions include:
1) aliphatic side chains: glycine, alanine, valine, leucine and isoleucine
2) aliphatic hydroxyl side chains: serine and threonine
3) amide-containing side chains: asparagine and histidine
4) aromatic side chains: phenylalanine, tyrosine and tryptophane
5) basic side chains: lysine, arginine and histidine
6) acidic side chains: aspartate and glutamate, and
7) sulfur-containing side chains: cysteine and methionine.

Preferred conservative amino acid substitution groups are: valine-leucine-isoleucine, phenylalanine-tyrosine, lysine-arginine, alanine-valine, glutamate-aspartate, and asparagine-glutamine.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between wild type protein and mutant protein thereof. Polypeptide sequences also can be compared using FASTA with default or recommended parameters; a program GCG version 6.1 FASTA (e.g., FASTA2 and FASTA3) provides alignments and percent sequence identity of the regions of the best overlap between the query and search sequences. Another preferred algorithm when comparing a sequence of the invention to a database containing a large number of sequences from different organisms is the computer program BLAST, especially BLASTP or TBLASTN, using default parameters.

The term “sequence identity” when referring to a nucleic acid or antigen-binding fragment thereof, indicates that, when optimally aligned with appropriate nucleotide insertions or deletions with another nucleic acid (or its complementary strand), there is nucleotide sequence identity in at least about 90%, and more preferably at least about 95%, at least about 96%, at least about 97%, at least about 98% or at least about 99% of the nucleotide bases, as measured by any well-known algorithm of sequence identity, such as FASTA, BLAST or GAP as mentioned above.

As applied to polypeptides, the term “sequence identity” indicates two peptide sequences, when optimally aligned, such as by the programs GAP or BESTFIT using default gap weights, that share at least 90%, even more preferably at least 95%, at least 98% or at least 99% sequence identity. Preferably, residue positions which are not identical differ by conservative amino acid substitutions.

The term “affinity” as used herein means that an antibody, or antigen-binding fragment thereof, or a synthetic and biotechnological derivative thereof forms a complex with an antigen that is relatively stable under physiologic conditions. Affinity can be characterized by an equilibrium dissociation constant (K_{D}) of less than 1×10⁻⁵ M, preferably less than 1×10⁻⁶ M, more preferably less than 1×10⁻⁷ M (a smaller K_{D} denotes a tighter binding). Affinity of an antibody is a measure of the strength of the binding between antibody and antigen, such that a low-affinity antibody binds weakly and a high-affinity antibody binds firmly.

The term specifically binds” or “binds specifically” or the like means that an antibody, or antigen-binding fragment thereof, or a synthetic and biotechnological derivative thereof does recognize its antigen specifically and selectively. In comparison to affinity, antibody specificity is a measure of the goodness of fit between the antibody and its antigen, such that a low-specificity antibody shows cross-reactivity with other proteins and a high-specificity antibody shows no cross-reactivity with other proteins.

The term “K_{D}” as used herein, is intended to refer to the equilibrium dissociation constant of a particular antibody-antigen interaction. Methods for determining whether two molecules specifically bind are well known in the art and include, for example, equilibrium dialysis, surface plasmon resonance, e.g., BIACORE™, and the like.

The term “high affinity antibody” as used herein, refers to monoclonal antibodies having a binding affinity K_{D} to kynurenine of less than 10⁻⁵ M, preferably less than 10⁻⁶ M, more preferably less than 10⁻⁷ M (a smaller K_{D} denotes a tighter binding), as measured by surface plasmon resonance (BIACORE™).

The term “antigen” as used herein, refers to a molecule or a portion of a molecule capable of being bound by a selective binding agent, such as an antigen-binding protein (including e.g. an antibody or immunological functional fragment thereof). An antigen can possess one or more epitopes that are capable of interacting with different antigen-binding proteins, e.g., antibodies.

The term “surface plasmon resonance” as used herein, refers to an optical phenomenon that allows for the analysis of real-time bio specific interactions by detection of alterations in protein concentrations within a biosensor matrix, for example using the BIACORE™ system.

The term “compete” when used in the context of the antibody, or antigen-binding fragment thereof, or synthetic and biotechnological derivative thereof, means competition between the reference antibody in binding specifically to the target protein (also called “antigen”, in the specific case of the present invention this is KYN) with other antigen-binding molecules. Such antigen-binding molecules may be selected from the list of: natural ligands to KYN, other antibodies or antigen-binding fragments thereof, diabodies, nanobodies, anticalins, single-chain antibody variable regions etc. This competition may be determined by an assay in which the reference antibody prevents or inhibits specific binding of another antigen-binding molecule, such as for example a test-antibody, from binding specifically to KYN.

Numerous types of competitive binding assays can be used to determine if one antigen-binding protein competes with another, for example solid phase direct or indirect radioimmunoassay (RIA), solid phase direct or indirect enzyme immunoassay (EIA), sandwich assay, competition assay, or solid phase direct labeled assay.

The term “kynurenine(s)” as used herein, refers to the amino acid itself as well as to its esters and salts. Thus, kynurenine and/or its esters and salts are included in said term.

Embodiments of the present invention can provide a monoclonal antibody, or an antigen-binding fragment thereof, or a synthetic and biotechnological derivative thereof, that specifically binds kynurenine and acts as a detection marker for inflammation in an individual. Embodiments of the present invention also provides methods for the detection of a disease or condition in an individual using the inventive monoclonal antibody, or an antigen-binding fragment thereof, or a synthetic and biotechnological derivative thereof by determining KYN in a body fluid. Further, means in the form of test kits for performing the inventive method are provided. The tests can easily be performed by the individuals or patients on their own and at any given time at which such determination seems appropriate when saliva is used as the sample. Thus, embodiments of the invention can provide a reliable method to detect various diseases and conditions quickly and easily. It further provides a reliable method for the early measurement and prediction of inflammatory developments. This is beneficial for the therapy of the patient. The methods disclosed herein should be used together with clinical parameters. The relative value of kynurenine may preferably be interpreted together with other clinical parameters. Embodiments of the present invention can contribute contributes substantially to the prognostic value of a diagnosis. Embodiments of the method can be improved by comparing the value of kynurenine measured in the patient to be diagnosed with the average value obtained from a comparable cohort of persons who do not suffer from the disease.

Unless otherwise defined herein, scientific and technical terms used in connection with the present invention shall have the meanings that are commonly understood by those of ordinary skill in the art. Further, unless otherwise required by the context, singular terms shall include pluralities and plural terms shall include the singular. Generally, nomenclatures used in connection with and techniques of biochemistry, enzymology, molecular, and cellular biology, microbiology, genetics and protein and nucleic acid chemistry and hybridization described herein are those well-known and commonly used in the art. It has also to be noted that aspects of the present invention have been described with reference to different subject-matters. In particular, some aspects have been described with reference to method type claims, whereas other aspects have been described with reference to use type claims and/or product type claims, respectively. However, a person skilled in the art will gather from the above and the following description that, unless otherwise notified, in addition to any combination between features belonging to one type of subject-matter, also any combination relating to different types of subject-matter is considered to be disclosed with this text. In particular, combinations between features relating to method type claims and features relating to use type claims and/or product type claims are considered to be disclosed. It should additionally be noted that the term “comprising” does not exclude other elements or steps. Further, elements described in association with different embodiments may be combined.

### BRIEF DESCRIPTION OF THE DRAWINGS

The invention and embodiments thereof will be described below in further detail in connection with the drawing(s).

FIG. 1 shows a schematic overview of the kynurenine pathway.

FIG. 2 shows the chemical structure of kynurenine.

FIG. 3a shows a correlation of kynurenine concentrations in serum and saliva samples in healthy individuals.

FIG. 3b shows the level of KYN in serum and saliva samples.

FIG. 4 shows the difference of KYN-levels in healthy and diagnosed patients suffering from different diseases.

FIGS. 5a and 5b, respectively, show the results of a titration ELISA of the antibody of an exemplary embodiment of the present invention YU552-A01.

FIG. 6 shows the results of a competitive ELISA of the antibody of an exemplary embodiment of the present invention YU552-A01.

FIG. 7 shows an affinity measurement of the inventive antibody using surface plasmon resonance.

FIG. 8 shows an affinity measurement of an anti-KYN antibody of the prior art using surface plasmon resonance.

FIG. 9 shows a schematical display of the phage display technique.

FIG. 10 shows a table of KYN-level measurements using prior art antibodies against KYN.

FIG. 11 shows the results of an ELISA test of serum samples using the inventive antibody YU552-A01.

FIG. 12 shows the results of a LFA test of saliva samples using the inventive antibody YU552-A01.

### DETAILED DESCRIPTION

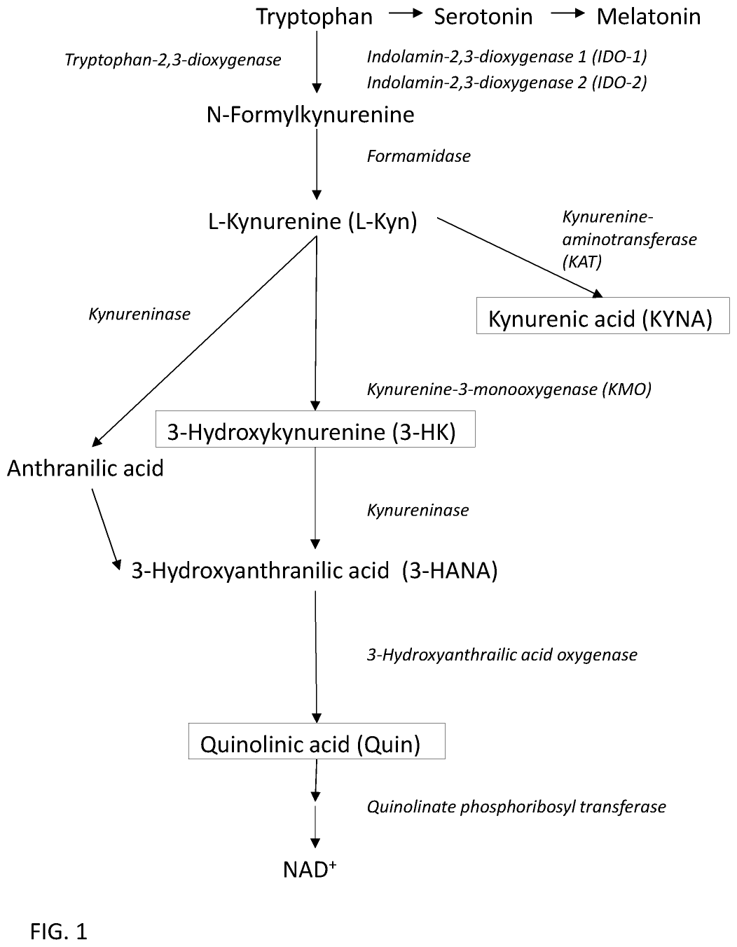

In FIG. 1, the kynurenine pathway (KP) is depicted which is the major route of the tryptophan degradation in higher eukaryotes. Enzymes are indicated in italics. Tryptophan is an essential amino acid that can be metabolized through different pathways, a major route being the KP. The first enzyme of the pathway, indoleamine-2,3-dioxygenase, IDO-1, is strongly stimulated by inflammatory molecules, particularly interferon-γ. Thus, the KP is often systematically up-regulated when the immune response is activated. The biological significance is that the depletion of tryptophan and generation of kynurenines play a key modular role in the immune response. It was found surprisingly by the inventors that the level of kynurenine measured in a body fluid can be used for the detection of inflammation in an individuum.

The central intermediate of the KP is L-kynurenine, L-KYN, where the metabolic pathway divides into two different branches. L-KYN is transformed to either the neuroprotective kynurenic acid, KYNA, via kynurenine aminotransferase, KAT, or the neurotoxic 3-hydroxykynurenine, 3-HK. 3-HK is further metabolized in a sequence of enzymatic steps to finally yield NAD⁺.

An increase in kynurenine is important in inflammatory metabolism for the body to produce an anti-inflammatory. Kynurenine can pass the blood-brain barrier and thus enter the brain. A high conversion of TRP to kynurenine therefore also carries an increased risk of neuroinflammatory or neurotoxic damage.

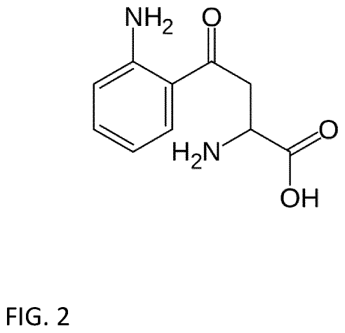

FIG. 2 shows the chemical structure of the amino acid KYN. KYN is a very small molecule (molecular weight 209) and is synthesized by the enzymes TDO and IDO-1 in the kynurenine pathway (KP) described above. The chemical formula of KYN is C₁₀H₁₂N₂O₃.

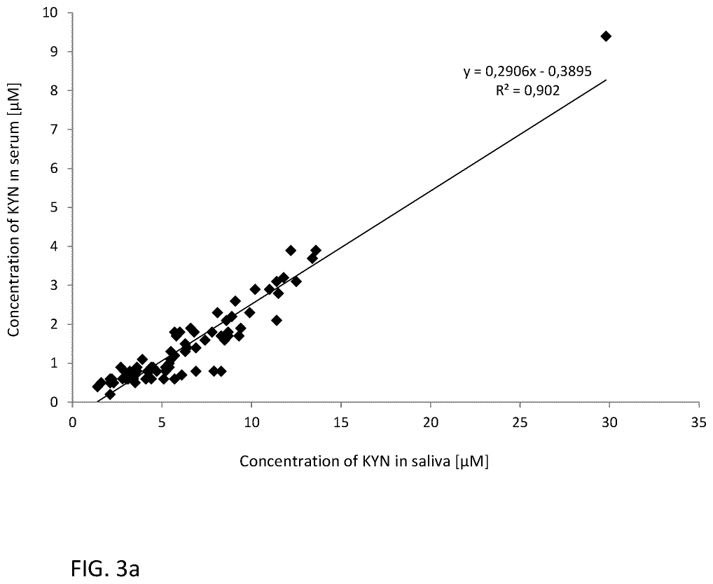

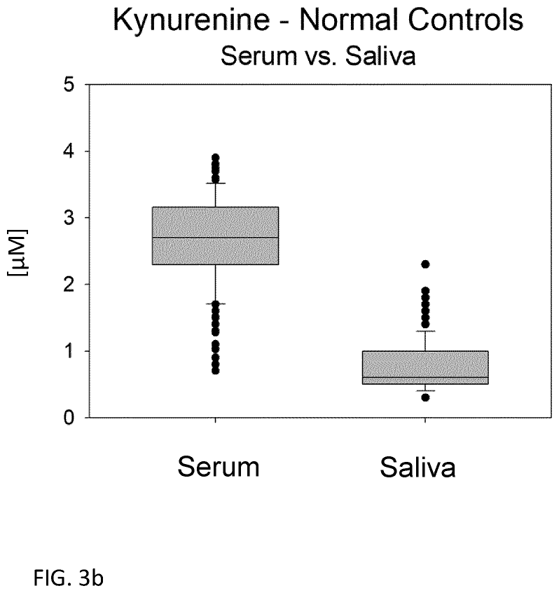

FIG. 3a shows the results of measurements of the concentration of KYN in serum and saliva samples. The inventors of the present invention were able to determine a reliable relation between kynurenine levels in serum and saliva using a color reaction. There exists a linear correlation (R2=0.902) between the measurement of kynurenine in serum and saliva. The relation is 3.8 (serum): 1 (saliva), which is not the case for many other proteins.

In terms of the coloring reaction, before the content of kynurenine can be determined, components which may negatively affect the correct and precise test result have to be removed. Said undesired components of the sample which may disturb the correct test result are removed preferably by precipitation. Such precipitation can preferably be performed by using trichloric acid. It is, however, possible to use other methods for deproteinization of the components of the sample than using trichloric acid. After the disturbing components of the sample have been removed by precipitation, it may be necessary to separate the phases by centrifugation. The supernatant is then preferably reacted with a coloring reagent which may preferably be Ehrlich's reagent. After development of the color, the samples are measured by measuring the absorbance at a suitable wavelength. Preferably, the test is performed in a quantitative or semi-quantitative manner. In the test method, either a calibration curve can be used, or a certain threshold value is fixed in the test kit in order to avoid false positive results.

As already mentioned above, it was found by the inventors that the concentration of KYN measured in μM either in saliva (x-axis) or in serum (y-axis) using a color reaction does correlate. This was surprising because not all components that can be detected in serum are also detectable in saliva. The inventive method is based on the surprising finding of the inventors that the level of kynurenine in especially saliva corresponds proportionally very well with the level of kynurenine in serum samples. Accordingly, embodiments of the method of the present invention can be preferably performed with saliva.

The determination of kynurenine is preferably performed quantitatively or semi-quantitatively since it is important to detect changes of the level of kynurenine which are outside the regular range. It is particularly advantageous that the in vitro method can be performed without a physician or any other trained people when using saliva as a sample.

Saliva is a clear, slightly acidic and complex body fluid. Like blood, saliva contains a variety of enzymes, hormones, antibodies, antimicrobial constituents, and growth factors. Many of these components enter saliva from the blood by passing through the spaces between cells. Therefore, most compounds found in blood are also present in saliva. Thus, saliva is functionally equivalent to serum on reflecting the physiological state of the body, including emotional, hormonal, nutritional and metabolic variations. However, although saliva contains diverse components with diagnostic properties, there are some major limitations which have prevented people in the art from recognizing the full potential of disease detection by using this body fluid. There is, for example, the low concentration of the components compared with levels in the blood, the lack of definitive biomarkers for specific diseases, the lack of an easy and inexpensive sampling method with minimal discomfort, and the lack of an accurate, easy-to-use, and portable platform to facilitate (early) disease detection. Furthermore, another problem of using saliva as a sample lies in the reproducibility of saliva tests.

This shows that saliva cannot be used for determining every possible parameter. Saliva measurements of the glucose level of patients having diabetes, e.g., are not successful so far. The same is true for the detection of different viruses in saliva. This became also evident in the Corona pandemic when various saliva tests failed to detect the causative agent of Corona, SARS-CoV-2. Consequently, saliva cannot simply be used and measurements in saliva are not self-explanatory. This applies to the choice of matrix (serum, plasma, or saliva) as well as to the methodology and finally the chosen technique.

FIG. 3b shows the results of KYN-measurements of serum and saliva samples of 304 healthy individuals, being the normal control. The measurements were performed using the color reaction. As can be seen from the graph, the relationship of KYN in serum to saliva is 3.8:1. Thus, the level of KYN is significantly lower in saliva compared to serum.

As already described above, the method to detect KYN used preferably today is HPLC. This is due, among other things, to the fact that KYN is a very small molecule, which makes it per se difficult to produce antibodies that recognize said molecule. Furthermore, KYN is immunoactive, and thus inhibits antibody production. In addition, the inventors were able to show that the level of KYN in saliva is significantly lower than in serum, which adds to the difficulties to produce an antibody that is able to detect KYN also in body fluids that show low KYN-levels, like saliva. Due to the low occurrence of KYN in body fluids such as saliva, it is very important for an antibody directed against KYN to be highly specific and sensitive for the antigen in order to detect this amino acid in a body fluid like saliva. Thus, especially antibodies against KYN need to have a high specificity for its antigen.

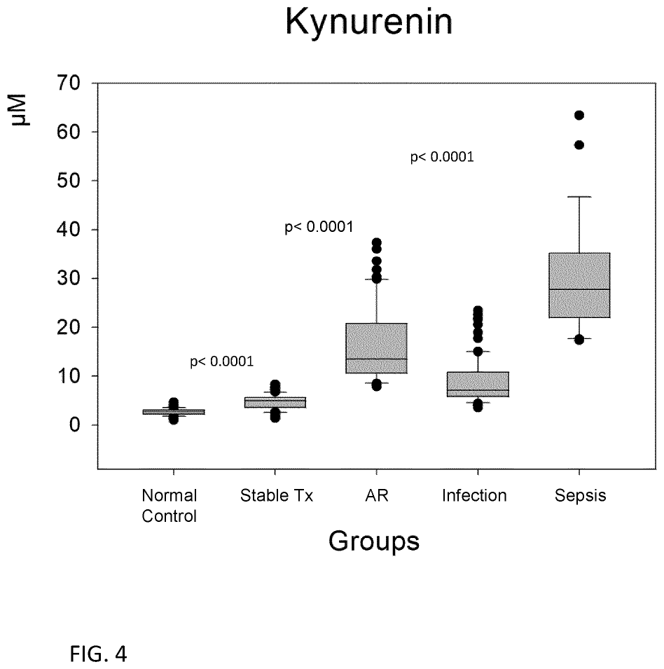

FIG. 4 depicts the results of a study to examine KYN-levels in patients suffering from different diseases. In this study, 304 normal controls (healthy persons), 314 patients after renal transplantation in stable condition (stable Tx), 96 patients after renal transplantation with an acute rejection (AR), 274 patients with an infection, and 48 patients suffering from sepsis have been included. The measurements were performed with serum samples using the color reaction.

As can be seen from the graph, stably transplanted individuals show an only slightly increased KYN-level compared to healthy persons. This is due to the sufficient immunosuppression and a corresponding down-regulated innate immune response in stably transplanted individuals. Thus, there is only very little inflammation in this group. In contrast thereto, individuals having an activated immune system, which is the case in transplanted patients having an acute rejection, in infected patients, as well as in patients suffering from a sepsis, do show a significantly elevated KYN-level in comparison to KYN-levels of healthy individuals.

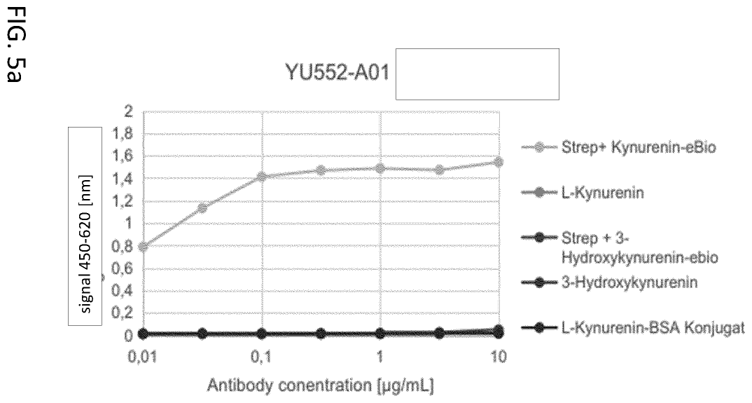

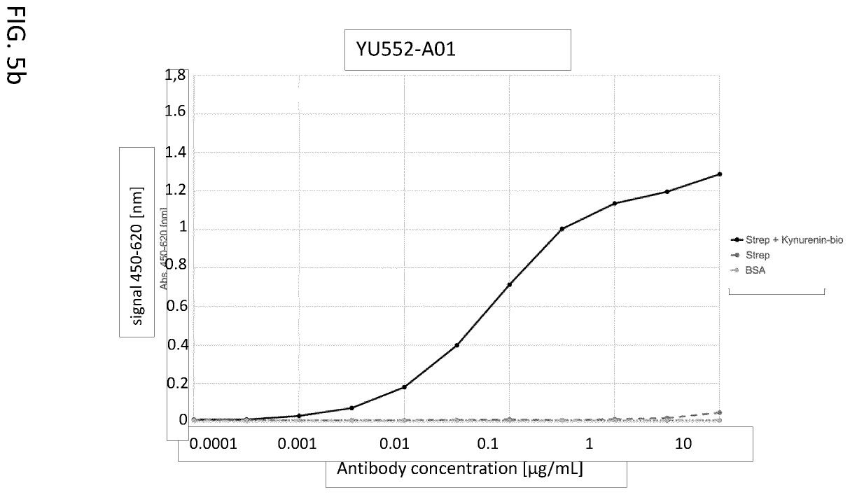

FIGS. 5a and 5b, respectively, show a titration ELISA. This assay is generally used to test specific binding of an antibody to its antigen. In a titration ELISA, an antigen at a constant concentration is titrated with different concentrations of added antibody. The antigen is immobilized to a solid surface, and after binding to the antigen, free antibody is removed. Bound antibody remains with the antigen, which is immobilized to the surface of the microtiter well. The amount of bound antigen is quantified with detection antibodies, which provide a detectable signal.

The inventive antibody YU552-A01 showed specific binding to biotinylated KYN (Strep+Kynurenin-eBio), which was used for selection of the antibody. No binding could be detected to the other coated antigens (FIG. 5a) and to the conjugates streptavidin and bovine serum albumin (BSA) (FIG. 5b). Thus, the inventive antibody is highly specific for its antigen KYN and does not show any cross-reactivity.

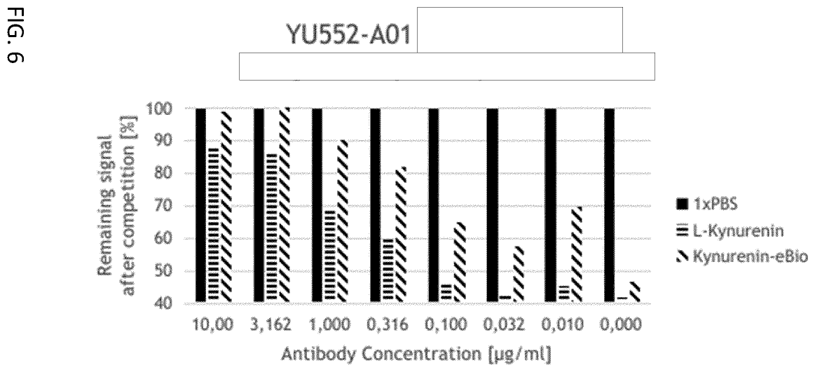

In FIG. 6, the results of a competitive ELISA are depicted. Generally, there are different configurations of ELISA tests known. ELISA types are direct ELISA, sandwich ELISA, competitive ELISA and/or reverse ELISA. Usually, a so-called sandwich ELISA is performed. In such an ELISA test, the compound that binds specifically to the analyte is fixed on a solid surface (e.g., the bottom of a microtiter well). Unspecific binding sites are saturated (e.g., with skim milk powder) in order to avoid unspecific binding. Usually, several microtiter wells are coated with the component in order to allow an easy dilution of the sample for a determination of the content of the analyte. The binding of the analyte to the relevant wells is usually detected with another antibody that binds, however, to another area of the target molecule in order to avoid negative interference of the binding. Such an antibody is usually coupled with a signal generating means that may be for example an enzyme like horseradish peroxidase (HRP). The presence of the analyte to be detected can then be seen by adding a precursor molecule which is converted to another molecule having different properties by the signal generating molecule. When, for example, kynurenine is present in one well, the antibody binds to this molecule and a color signal is generated with the activity of the signal generating means (e.g., HRP), whereby the intensity is proportional to the amount of the bound target molecule. The reaction can be measured quantitatively and the amount of the analyte to be detected in the body fluid can be determined precisely.

Another type of ELISA test is a competitive ELISA, which is performed by immobilizing the compound that binds the analyte on a solid surface and adding the sample to be analyzed including the analyte, in an exemplary embodiment of the present invention KYN, to bind to the compound, being the inventive antibody YU552-A01. The less analyte is present in the sample, the more free binding sites on the compound are there. These free binding sites are bound by a synthetic, marked analyte which is being made visible by a color reaction. Thus, the less analyte is present in the sample, the more synthetic, marked analyte is bound to the compound, and the stronger is the signal. Conversely, this means that the more KYN is in the sample and binds to the inventive antibody, the less signal is detectable. The detection of the analyte and the measurement of its concentration is indirectly possible via the detection and, if necessary, a quantitative determination of the labelling.

Thus, competitive inhibition is measured by determining the amount of label in the presence of the compound. Usually, the compound is present in excess.

Typically, when a competing compound is present in excess, it will inhibit specific binding of a reference antigen-binding protein to a common antigen by at least 40%, at least 45%, at least 50%, at least 55%, at least 60%, at least 65%, at least 70%, or at least 75% or more. In some instances, binding is inhibited by at least 80%, at least 85%, at least 90%, at least 95% or at least 97% or more.

As can be seen in FIG. 6, the inventive antibody YU552-A01 showed a reduced ELISA binding signal in the presence of the conjugated target antigen KYN-ebio compared to the negative control phosphate buffered saline (PBS). The non-conjugated, free target antigen L-KYN showed an even more reduced ELISA signal, which means that more of the non-conjugated, free KYN binds to the inventive antibody. This blocking effect is better detectable at lower antibody concentrations as is apparent from FIG. 6. Thus, the inventive antibody binds to the antigen KYN itself and shows no cross-reactivity to the conjugate biotin. These results show that the antibody that can be used in embodiments of the invention recognizes KYN itself and not the linker biotin. The results thus also confirm the high specificity of the inventive antibody to KYN.

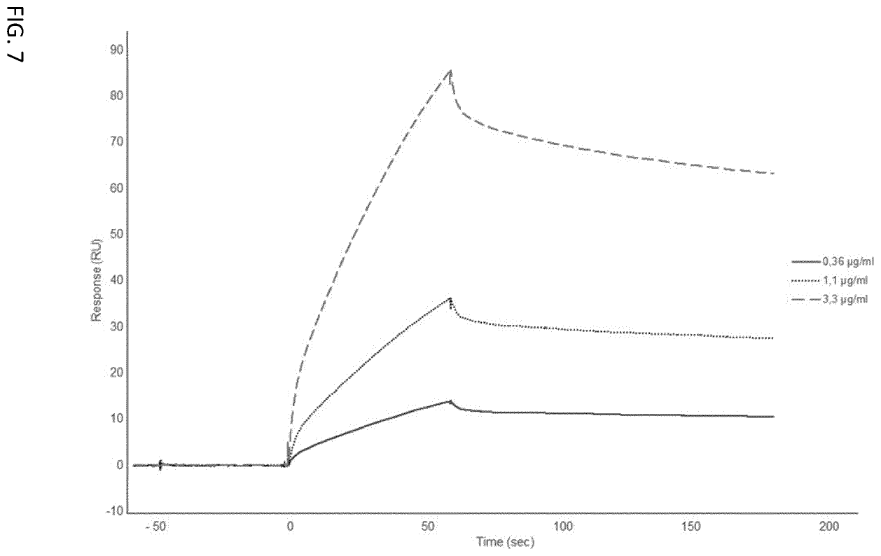

In FIG. 7, the results of a surface plasmon resonance measurement performed with the inventive antibody YU552-A01 are shown. Surface plasmon resonance spectroscopy (SPR spectroscopy) is a spectroscopic analysis method that measures the refractive index of an analyte with very high resolution. The advantage of this method is that the binding kinetics can be traced in real time without influencing the analytes. For the determination of protein interactions, a membrane is prepared on a prism with a gold film that can adsorb biological molecules. Covering the membrane with molecules changes the refractive index of the layer, which can be measured very sensitively with this method.

For the SPR, the antibody was used in different concentrations as is depicted in FIG. 7. As is apparent from the graph, binding of the antibody YU552-A01 to free, not immobilized KYN could be detected.

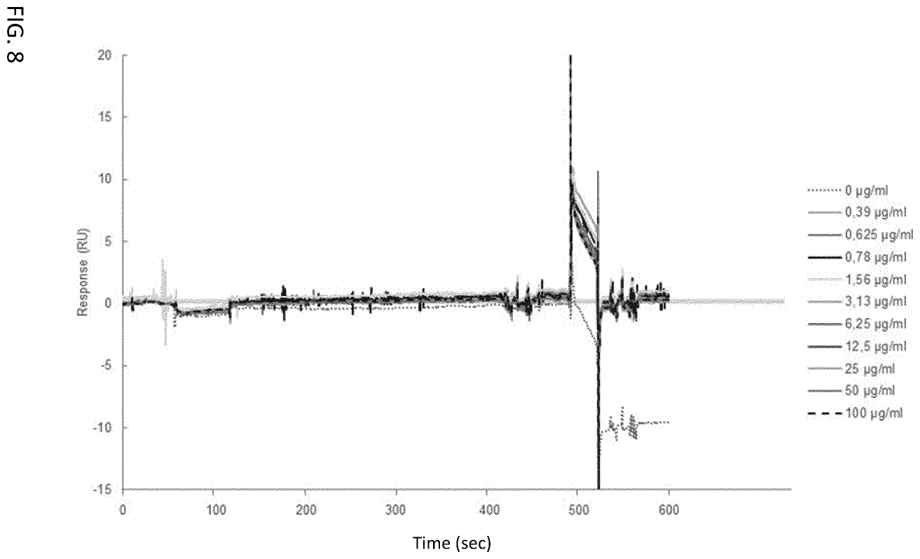

FIG. 8 shows the results of an SPR spectroscopy with a KYN-antibody of the prior art. The antibody sc-69890 by the company Santa Cruz was used as the prior art antibody, as this antibody is still commercially available. Said antibody is a mouse monoclonal antibody being directed against N-Formyl-KYN. As can be seen from the graph, the prior art antibody is not able to bind free KYN. The exemplary embodiment of an antibody of the present invention and the antibody of the prior art were produced by different manufacturing processes. As a result, they differ in their properties and structure. Accordingly, not every antibody produced against KYN shows the same properties in terms of specificity and affinity towards the antigen KYN. This also shows the need for an antibody that reliably recognizes KYN in different body fluids, preferably saliva.

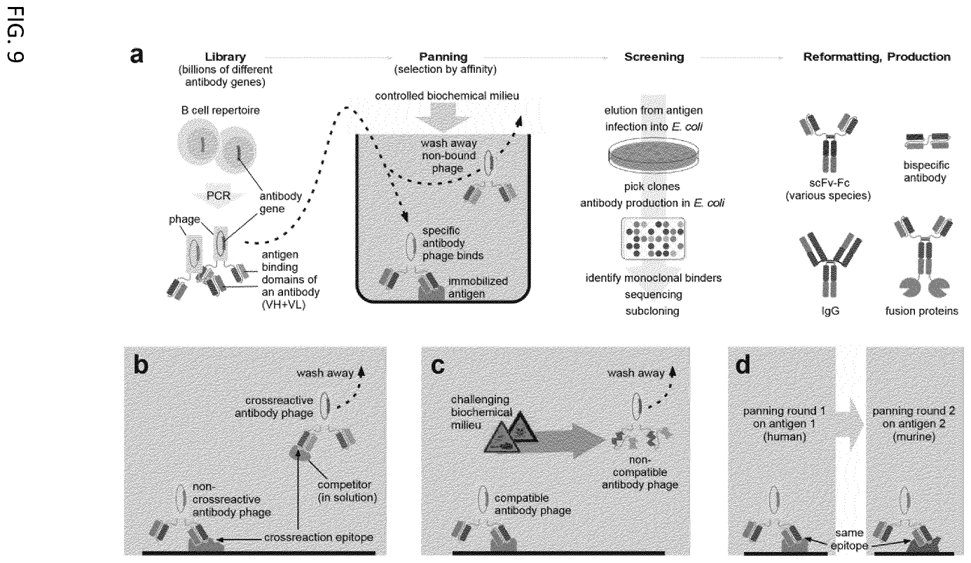

In FIG. 9, the phage display technique is depicted. In general, isolation of fully human or humanized monoclonal antibodies can be conducted in different ways. In one approach, a mouse is used in which the immunoglobulin genes have been replaced with human genes. This mouse then makes antibody responses that use human antibody sequences. A second approach uses a bacteriophage display library of human variable region sequences and selects for antigen-binding in vitro. The selected genes are then combined with human constant region genes to reconstitute a complete IgG antibody. A third approach utilizes B-cells to produce antibodies. As will be outlined in the examples in more detail, suspension panning may be used for high throughput antibody identification.

The exemplary embodiment of an antibody of the present invention was produced using the phage display method. In principle, phage display is a laboratory technique for the study of protein-protein, protein-peptide, and protein-DNA interactions that uses bacteriophages (viruses that infect bacteria) to connect proteins with the genetic information that encodes them. In this technique, a gene encoding a protein of interest is fused to a phage coat protein gene, causing the phage to “display” the protein on its outside while containing the gene for the protein on its inside, resulting in a connection between genotype and phenotype. These displaying phages can then be screened against other proteins, peptides, or DNA sequences in order to detect interaction between the displayed protein and those other molecules. In this way, large libraries of proteins can be screened and amplified in a process called in vitro selection which is analogous to natural selection. There are three categories of phage display:
1. phage display of antibody libraries,
2. phage display of peptide libraries,
3. phage display of cDNA libraries.

The scheme of an antibody phage display is illustrated in FIG. 9. In an antibody phage particle, the antibody gene and the function it encodes (antigen binding) are physically linked. This allows the affinity selection of monoclonal human antibodies in the test tube (a process named ‘panning’). Panning can be performed in the presence of a soluble competitor to deplete cross-reacting antibodies. Panning under defined biochemical conditions selects only antibodies that are functional at these conditions. Sequential panning rounds on two different but homologous antigens are also possible, allowing functional selection of antibodies that bind to a structural feature common to the two proteins. After panning, screening for monoclonal antibodies against the used antigen is performed, subsequently followed by production of said antibodies.

Antibodies developed by phage display can be produced in high yield in E. coli or other cell systems. This is a major advantage to antibodies that are produced in animals.

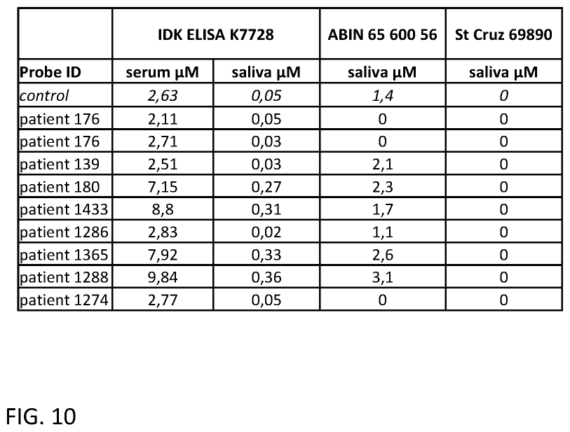

FIG. 10 shows the results of a measurement of various prior art antibodies that are directed against KYN, in detecting KYN in samples of nine patients after renal transplantation and one healthy control person using an ELISA test. The measurements were performed in serum and/or saliva.

The prior art antibodies used in the measurement are:

IDK ELISA K7728: polyclonal antibody against L-KYN included in an ELISA kit.

ABIN 6560056: mouse monoclonal antibody against L-KYN.

St. Cruz 69890: mouse monoclonal antibody against N-Formyl-KYN.

So far, due to the above-mentioned difficulties to produce antibodies that bind KYN, there are only a few antibodies against KYN described in the art which not all are commercially available anymore. None of them have been produced by phage display but in different animals. Further, none of these antibodies are developed for LFA tests. As is common knowledge, it is not possible to simply switch between test systems for which antibodies have not been developed. Thus, the antibodies of the prior art could not be tested in another test system like LFA. However, to be able to test the specificity of the prior art antibodies, ELISA tests were performed using serum and/or saliva samples of patients after renal transplantation either stably transplanted (patient 176), or showing an acute rejection (patients 139, 180, 1433, 1286, 1365, 1288, 1274), and in a healthy control person.

As already mentioned above, the level of KYN in a patient is at least two times higher than in the healthy control group. It was not possible to detect this relationship reliably in the tested samples using the antibodies of the prior art, not in the serum samples and even more pronounced in the saliva samples. One antibody did not detect KYN at all in saliva.

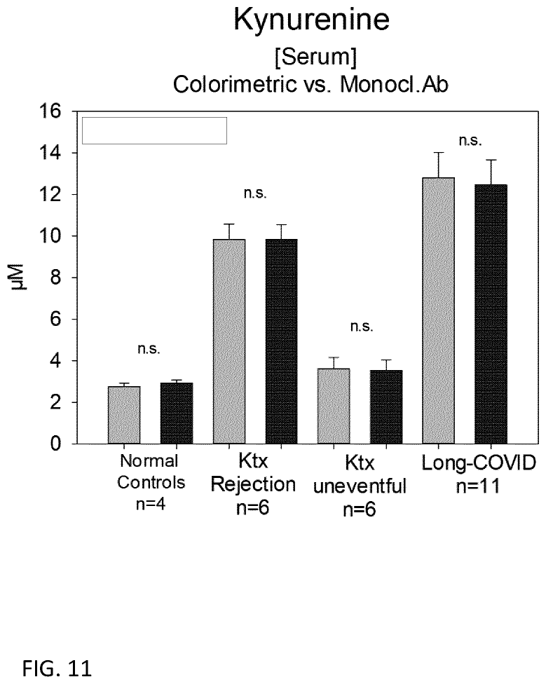

The results demonstrate the difficulty in producing an antibody that reliably recognizes KYN even in body fluids with low KYN levels, like saliva. FIG. 11 shows the results of a competitive ELISA test of serum samples using the exemplary embodiment of an antibody of the present invention YU552-A01. As can be seen from the graph, the exemplary embodiment of an antibody of the present invention is able to detect KYN in serum samples. The grey bars indicate the results of the colorimetric measurement depicted in Example 4 and also referred to in FIG. 4. In comparison, the black, dotted bars indicate the results of the ELISA tests using the inventive antibody YU552-A01. The inventors were able to show that the exemplary embodiment of an antibody of the present invention is as reliable as the colorimetric measurement in detecting KYN in serum samples. As is also apparent from the graph, the exemplary embodiment of an antibody of the present invention is able to detect KYN in different patient samples.

Abbreviations/terms of FIG. 11:
Ktx rejection=samples of patients after a kidney transplantation showing an acute rejection
Ktx uneventful=samples of patients after a kidney transplantation showing stable conditions
Long-COVID=samples of patients suffering from the Long-COVID syndrome after an infection with the SARS-CoV-2 virus causing a Corona infection
n=number of samples, i.e. patients, included in the measurement
n.s.=not specific
Monocl. Ab=monoclonal antibody
Colorimetric=colorimetric measurement

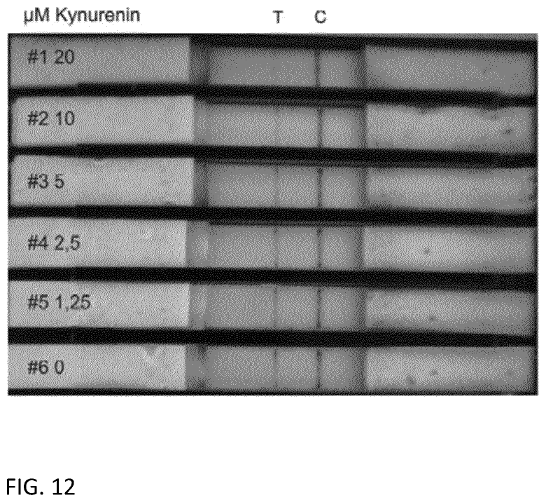

FIG. 12 shows a photograph of a LFA test of saliva samples using the exemplary embodiment of an antibody of the present invention. LFA tests are simple devices intended to detect the presence (or absence) of a target analyte without the need for specialized and costly equipment, though many lab-based applications exist that are supported by a reading equipment. Typically, these tests are used for medical diagnostics either for home testing, point of care testing, or laboratory use. A widely spread and well-known application is the home pregnancy test, as well as the recently widely used “Corona test”. The technology is based on a series of capillary beds, such as pieces of porous paper or sintered polymer. Each of these elements has the capacity to transport fluid (e.g., saliva) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles in a salt-sugar matrix that contains everything to guarantee an optimized chemical reaction between the target molecule and its chemical partner that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and the technology worked fine, the second contains a specific capture molecule and only captures those particles onto which an analyte molecule has been immobilized. After passing these reaction zones, the fluid enters the final porous material, the wick, that simply acts as a waste container. Lateral Flow Tests can operate as either competitive or sandwich assays.

In principle, any colored particle can be used. However, latex (blue color) or nanometer sized particles of gold (red color) are most commonly used. The gold particles are red in color due to localized surface plasmon resonance. Fluorescent or magnetic labeled particles can also be used. However, these require the use of an electronic reader to assess the test result. The sample first encounters colored particles which are labeled with antibodies raised to the target analyte. The test line will also contain antibodies to the same target, although it may bind to a different epitope on the analyte. The test line will show as a colored band in positive samples. While not strictly necessary, most test kits preferably incorporate a second line which contains an antibody that picks up free latex/gold in order to confirm the test has operated correctly.

The LFA tests shown in FIG. 12 are competitive assays. As already explained above, in a competitive assay, an antibody specific for the antigen is bound (immobilized) to a solid phase. The sample solution containing the analyte and a second solution (tracer solution) containing the antigen to be detected in a known concentration and labelled with a detection system are added. During the reaction time (incubation time), the unlabeled antigen present in the sample solution in unknown concentration and the labelled antigen present in known concentration compete for the binding sites of the immobilized antibody. Consequently, the higher the concentration of antigen from the sample, the less labeled antigen is bound to the antibody. The detection of the antigen and the measurement of its concentration is indirectly possible via the detection and, if necessary, a quantitative determination of the labelling. Thus, the less signal there is, the more unlabeled KYN of the sample binds to the inventive antibody YU552-A01.

In the depicted LFA test, commercially available saliva samples (Invent Human Specimen) were used. KYN in different concentrations (0 μM, 1.25 μM, 2.5 μM, 5 μM, 10 μM and 20 μM) was added to ensure that enough KYN was available for the exemplary embodiment of an antibody of the invention to bind, thus for the assay to work.

As is apparent from the photograph taken of the different LFAs showing the various KYN concentrations, the antibody YU552-A01 is able to detect KYN in saliva samples. Due to the fact that unlabeled KYN competes with labelled antigen for binding to the exemplary embodiment of an antibody of the present invention, the signal decreases as the concentration of KYN increases because more unlabeled KYN binds to the inventive antibody. The signal in the sample with no added KYN is based on the fact that KYN is always present in saliva of a human sample.

As could be shown, exemplary embodiments of the present invention can provide a reliable method and test kit for the detection of inflammation by determining the level of kynurenine in a body fluid, preferably saliva. Using the inventive monoclonal antibody, the inventors provide a tool to determine the level of KYN easily and quickly without the need of any trained person when using saliva as the sample. This is also beneficial in pediatric medicine and monitoring of various diseases.

The present invention is further illustrated by the following examples which are, however, not limiting the scope of the present invention.

### Example 1: Discovery of Monoclonal Antibodies

As already mentioned above, for the identification of antibodies for embodiments of the present invention, a phage display technique was applied. As libraries, a naive scFv library was used. As the antigen, L-Kynurenine and 3-Hydroxy-DL-kynurenine, both from Sigma Aldrich, were used. Biotinylated kynurenine, kynurenine alone, streptavidin plus biotinylated kynurenine and kynurenine-BSA were used as immobilized targets. As counter selection, streptavidin, IVIGs (intravenous immunoglobulin) and N protein standard (Siemens Healthcare) were used.

In total, 384 clones from lambda and kappa selection were isolated. 29 sequence clusters with binding antibodies were identified. The number of clones was reduced for IgG conversion based on a rational approach:

### 1.1 Antibody Selection Using Phage Display

The antibody selection was performed as follows: For the panning procedure, the antigen was immobilized on a Costar High binding 96 well plate (Corning). 1 μg of the target was diluted in phosphate buffered saline (PBS, 8 g/l NaCl, 0.2 g/l KCl, 1.8 g/l Na₂HPO₄*2 H₂O, 0.24 g/l KH₂PO₄) and coated in the wells at 4° C. overnight. Next, the wells were blocked with 350 μl BSA PBST (2% (w/v) bovine serum albumin, BSA in PBS; 0.05% Tween20) for 1 h at RT (room temperature) and then washed 3 times with PBST (PBS; 0.05% Tween20).

For immobilization of the biotinylated antigen, 2 μg Streptavidin in PBS was immobilized as described above. After blocking of the wells, 1 μg biotinylated antigen in 200 μl PBS was immobilized for 1 h and then washed three times with PBST.

Before adding the libraries to the coated wells, the libraries (5×10¹⁰ phage particles) were preincubated with 2% BSA-PBST (and 5 μg Streptavidin for panning on biotinylated antigen) on blocked wells for 1 h at RT. The libraries were transferred to the coated wells, incubated for 2 h at RT and washed 10 times. Bound phage was eluted with 200 μl trypsin (10 μg/ml) at 37° C. for 30 min and was used for the next panning round.

The eluted phage solution was transferred to a 96 deep well plate (Greiner Bio-One, Frickenhausen, Germany) and incubated with 190 μl E. coli TG1 (OD₆₀₀=0.5) firstly for 30 min at 37° C., then 30 min at 37° C. and 650 rpm to infect the phage particles. 4 ml 2×YT-GA (1.6% (w/v) Tryptone; 1% (w/v) yeast extract; 0.5% (w/v) NaCl (pH 7.0), 100 mM D-Glucose, 100 μg/ml ampicillin) was added and incubated for 1 h at 37° C. and 650 rpm, followed by addition of 5×10¹⁰ cfu (colony forming units) M13KO7 helper phage. Subsequently, the infected bacteria were incubated for 30 min at 37° C. followed by 30 min at 37° C. and 650 rpm before centrifugation for 10 min at 3220×g.

The supernatant was discarded, and the pellet resuspended in fresh 2×YT-AK (1.6% (w/v) Tryptone; 1% (w/v) Yeast extract; 0.5% (w/v) NaCl (pH 7.0), 100 μg/ml ampicillin, 50 μg/ml kanamycin). The phage antibodies were amplified overnight at 30° C. and 650 rpm and used for the next panning round. In total, three panning rounds were performed. In each round, the stringency of the washing procedures was increased (20× in panning round 2, 30× in panning round 3). In the third round XL1 Blue cells were used for infection as described for TG1. After the third panning round, the titer plate was used to select monoclonal antibody clones for the screening ELISA.

1.2 Screening of Monoclonal Recombinant Binders Using E. coli scFv Supernatant

Soluble antibody fragments (scFv) were produced in 96-well microtiter plates (MTP) with polypropylene (IJ96 PP, Greiner BioOne). 150 μl 2×YT-GA was inoculated with the bacteria bearing scFv expressing phagemids. MTPs were incubated overnight at 37° C. and 300 rpm, 70% humidity in a MTP shaker (SOK3190, Axon). A volume of 140 μl 2×YT-GA in a MTP well was inoculated with 10 μl of the overnight culture and grown at 37° C. and 300 rpm, 70% humidity until bacteria reached an OD600 of 0.5. Bacteria were harvested by centrifugation for 10 min at 3220×g and the supernatant was discarded. To induce expression of the antibody genes, the pellets were resuspended in 150 μl 2×YT supplemented with 100 μg/ml ampicillin and 50 μM isopropyl-beta D thiogalacto pyranoside (IPTG) and incubated at 30° C. and 300 rpm overnight.

Bacteria were pelleted by centrifugation for 10 min at 3220×g and 4° C. The scFv-containing supernatant was transferred to a new MTP and stored at 4° C. before ELISA analysis.

For the ELISA, 20 ng of antigen or 40 ng Streptavidin, followed by 20 ng biotinylated antigen after blocking was coated on 384 well microtiter plates (Greiner PS, F-bottom) in PBS (pH 7.4) overnight at 4° C. After coating, the wells were washed three times with PBST and blocked with 1% BSA-PBST for 1 h at RT, followed by three washing steps with PBST. Supernatants containing monoclonal scFv were mixed with 1% BSA-PBST (1:2) and incubated in the antigen coated plates for 1.5 h at RT followed by three PBST washing cycles. Bound scFv were detected using an anti-c-myc antibody (diluted in 2% BSA-PBST) and a goat anti-mouse serum conjugated with horseradish peroxidase (HRP) (A0168, Sigma) (1:50.000 dilution in 2% BSA-PBST).

Bound antibodies were visualized with tetramethylbenzidine (TMB) substrate (20 parts TMB solution A (30 mM 50 Potassium citrate; 1% (w/v) Citric acid (pH 4.1)) and 1 part TMB solution B (10 mM TMB; 10% (v/v) Acetone; 90% (v/v) Ethanol; 80 mM H₂O₂ (30%)) were mixed). After stopping the reaction by addition of 1 N H₂SO₄, absorbance at 450 nm with a 620 nm reference was measured in an ELISA plate reader (Epoch, BioTek). Monoclonal binders were sequenced and analyzed.

### 1.3 Characterization of the IgG in Titration ELISA

For titration ELISA, the antibodies were converted into mouse IgG2a format, expressed in HEK293 cells and purified. Purified IgG were titrated from 10 μg/ml-0.01 μg/ml on streptavidin+biotinylated KYN, spretavidin, KYN and KYN-BSA. The IgG were detected using goat-anti-mouse IgG (Fc)-HRP (A0168, Sigma).

### Example 2: Binding and Competitive Assays

Specific binding was tested by titration ELISA. Therefore, 200 ng Streptavidin in PBS was coated onto the wells of a 96 well plate (Costar 96 well plate, Corning #9018). Alternatively, 200 ng L-KYN, 3-Hydroxykynurenine and L-KYN-BSA conjugate diluted in PBS have directly been coated for 1 h at room temperature (RT). After 3 times washing with H₂O with 0.05% (v/v) Tween20 (H₂O-T), wells were blocked for 10 min with 300 μl/well 1% BSA-PBST and washed 3× with H₂O-T. Streptavidin coated plates were loaded with 200 ng biotinylated KYN or 3-Hydroxykynurenine for 1 h at RT and unbound antigen was removed by 3× washing with H₂O-T. Antibodies were diluted in 1/10 steps in 1×BSA-PBST, starting with 10 μg/ml. After washing (3×H₂O-T) 100 μl/well detection antibody was added for 1 h at RT (goat anti-mouse IgG (Fc specific), HRP coupled, Sigma #A0168). Unbound detection antibody was removed by additional washing with 3×H₂O-T and 100 μl TMB substrate (9.5 parts+0.5 parts TMB A (9.73 g/l potassium citrate, adjust to pH 4.1 with citric acid)+TMB B (90% (v/v) absolute EtOH, 10% (v/v) acetone, 2.4 g/L Tetramethylbenzidine, 0.907% (v/v) 30% H₂O₂) was added. Reaction was stopped by addition of 1 N H₂SO₄ and absorption was measured at 450 nm with a reference at 620 nm. Antibody YU552-A01 showed specific binding to biotinylated L-KYN whereas no binding could be observed to the other coated antigens.

For competition assays, biotinylated KYN was immobilized via Streptavidin on a 96 well plate (Costar 96 well plate, Corning #9018) and washed as described for the binding assay. Antibody YU552-A01 was pre-diluted to 10 μg/ml in PBS including 10 μg/ml KYN, biotinylated KYN and PBS without KYN, respectively. A serial 1/10 dilution of antibody and antibody KYN mixture was prepared in PBS and incubated for 15 min at RT. After washing of the coated plates with 3×H₂O-T, 100 μl antibody solutions were transferred into the coated wells and incubated for 1 h at RT. Unbound antibodies were removed by 3× washing with 300 μl H₂O-T and 100 μl/well detection antibody was added for 1 h at RT (goat anti-mouse IgG (Fc specific), HRP coupled, Sigma #A0168). Unbound detection antibody was removed by additional washing with 3×H₂O-T and 100 μl TMB substrate (9.5 parts+0.5 parts TMB A (9.73 g/l potassium citrate, adjust to pH 4.1 with citric acid)+TMB B (90% (v/v) absolute EtOH, 10% (v/v) acetone, 2.4 g/L Tetramethylbenzidine, 0.907% (v/v) 30% H₂O₂) was added. The reaction was stopped by addition of 1 N H₂SO₄ and absorption was measured at 450 nm with a reference at 620 nm.

Measured values were normalized to the ELISA signals obtained without the addition of competitor antigen (PBS). In general, a lower ELISA signal means more binding of the antigen.

### Example 3: Affinity Study

For affinity measurements by SPR spectroscopy, the inventive antibody was immobilized on CM7 chips and incubated with increasing concentrations of kynurenine and biotin-kynurenine. The measurements took place in a buffered aqueous system. For both analytes, 1:1 binding to the antibody could be determined. The affinity value for kynurenine is K_{D}: 2.09×10⁻⁵ M and for biotin-kynurenine K_{D} 1.27×10⁻⁵ M. The experiments were repeated with CM5 chips. In these experiments, the biotin-kynurenine was immobilized and incubated with increasing concentrations of inventive antibody. No affinity but only avidity could be determined for this experimental setup. This K_{D} value is 3.75×10⁻⁷ M.

The affinity measurements demonstrate that the antibody of embodiments of the present invention binds its antigen KYN strongly. Furthermore, it could also be shown that the antibody has no cross-reactivity and is thus very specific for its antigen KYN (FIGS. 5 and 6).

In contrast thereto, the prior art antibody sc-69890 by St. Cruz was tested as well for its affinity to KYN. This antibody was not able to bind non-immobilized biotin-KYN (FIG. 8).

These results show that antibodies which have been produced differently do possess different properties when it comes to binding its antigens, and thus, antibodies cannot be used interchangeably.

### Example 4: Color Reaction

### 4.1 General Approach

The tryptophan metabolites via kynurenine can be quantitatively determined in biologic fluids by color reactions which are known since many decades. In general, a detection method via the formation of a colored reaction product can be performed by standard methods.

Microplate Readers are laboratory instruments designed to detect biological, chemical or physical events of samples in microtiter plates. They are widely used in research, drug discovery, bioassay validation, quality control as well as manufacturing processes in the pharmaceutical and biotechnological industry and academic organizations. Sample reactions can be assayed in 6-1536 well format microtiter plates. The most common microplate format used in academic research laboratories or clinical diagnostic laboratories is a 96-well (8 by 12 matrix) with a typical reaction volume between 100 and 200 μL per well.

Common detection modes for microplate assays are absorbance, fluorescence intensity, luminescence, time-resolved fluorescence, and fluorescence polarization. Absorbance detection has been available in microplate readers for more than 3 decades, and is used for assays such as ELISA assays, protein and nucleic acid quantification or enzyme activity assays. A light source illuminates the sample using a specific wavelength (selected by an optical filter, or a monochromator), and a light detector located on the other side of the well measures how much of the initial (100%) light is transmitted through the sample: the amount of transmitted light will typically be related to the concentration of the molecule of interest.

### 4.2. Description of the Test

This test was developed as a modified method.

A color reagent was prepared and a dilution of a standard solution of kynurenine was also prepared. The color reaction is performed with a so-called “Ehrlich-Reagenz” which results in a yellow color. A solution comprising 2% by weight dimethylaminobenzaldehyde dissolved in 20% HCl is designated as “Ehrlich-Reagenz”. Said coloring reagent serves for the detection of primary amino groups, pyrrole and indole derivatives as well. The colorimetric determination of the concentration is performed with monochromatic light. The standard solution of kynurenine was prepared by using L-kynurenine sulfate.

Equal amounts of sample were mixed with 100 μl trichloroacetic acid (30%) thoroughly. After centrifugation, the supernatant was measured. The absorbents of each sample at 492 nm were compared with the absorbents at 650 nm or 690 nm of the same sample. Then the absorbents of the controls (average of 5 wells) were subtracted from the absorbents of each well. By preparing a standard curve, the concentration of kynurenine in each sample could be determined.

### Example 5: ELISA Test of Serum Samples Using the Antibody of an Embodiment of the Present Invention

Binding of KYN to the inventive antibody YU552-A01 was tested using a competitive ELISA. Serum samples of patients with different diseases and of a healthy individual were pre-treated with TCA according to the colorimetric measurement as depicted in Example 4 above. 50 μl/well serum samples were added into wells of a microtiter plate. YU552-A01 in a concentration of 0.25 μg/ml, 50 μl/well, was added, followed by shaking of the plate in a shaking table for 1 h at 170 rpm at RT. Then, the plate was washed three times using PBS-T, 250 μl/well, followed by another washing step using PBS, 250 μl/well, 1×. Then 100 μl/well HRP was added and incubated for 1 h at RT. Again, the plates were washed three times using PBS-T, 250 μl/well, and one time using PBS, 250 μl/well. 1-Step Ultra TMB, 100 μl/well was added, incubated for approx. 15-20 min, and the reaction was stopped using 2M H₂SO₄ (100 μl/well). OD was measured at 450 nm.

During the individual incubation steps, the plates or the corresponding rows were provided with a sealing foil to avoid any contamination.

1. A monoclonal antibody, or an antigen-binding fragment thereof, or a synthetic and biotechnological derivative thereof that specifically binds kynurenine and acts as a detection marker for inflammation in an individual.

2. The monoclonal antibody, or an antigen-binding fragment thereof, or a synthetic and biotechnological derivative thereof according to claim 1, that binds kynurenine in a body fluid.

3. The monoclonal antibody, or an antigen-binding fragment thereof, or a synthetic and biotechnological derivative thereof according to claim 1, wherein the monoclonal antibody, or an antigen-binding fragment thereof, or a synthetic and biotechnological derivative thereof comprises one or more complementarity-determining regions (CDRs), and/or a variable heavy chain (VH) region comprising an amino acid sequence having at least 90% sequence identity to the amino acid sequence of SEQ ID No: 1; and a variable light chain (VL) region comprising an amino acid sequence having at least 90% sequence identity to the amino acid sequence of SEQ ID No: 2.

4. The monoclonal antibody, or an antigen-binding fragment thereof, or a synthetic and biotechnological derivative thereof according to claim 1, having at least one complementarity-determining regions (CDR), is selected from:
heavy chain CDRs as defined by SEQ ID No: 3, SEQ ID No: 4, and SEQ ID No: 5, and
light chain CDRs as defined by SEQ ID No: 6, SEQ ID No: 7, and SEQ ID No: 8
and/or
wherein the monoclonal antibody, or an antigen-binding fragment thereof, or a synthetic and biotechnological derivative thereof comprises:
a heavy chain CDR1 (CDRH1) domain with an amino acid sequence as set forth in SEQ ID No: 3,
a heavy chain CDR2 (CDRH2) domain with an amino acid sequence as set forth in SEQ ID No: 4,
a heavy chain CDR3 (CDRH3) domain with an amino acid sequence as set forth in SEQ ID No: 5,
and
a light chain CDR1 (CDRL1) domain with an amino acid sequence as set forth in SEQ ID No: 6,
a light chain CDR2 (CDRL2) domain with an amino acid sequence as set forth in SEQ ID No: 7,
a light chain CDR3 (CDRL3) domain with an amino acid sequence as set forth in SEQ ID No: 8.

5. The monoclonal antibody, or an antigen-binding fragment thereof, or a synthetic and biotechnological derivative thereof according to claim 1, that is capable to specifically bind to kynurenine with an affinity (KD) of 10⁻⁵ M or less.

6. An isolated nucleic acid molecule encoding the monoclonal antibody, or antigen-binding fragment thereof, or synthetic and biotechnological derivative thereof according to claim 1.

7. An expression vector comprising the nucleic acid molecule according to claim 6.

8. A method of producing a monoclonal antibody, or an antigen-binding fragment thereof, or a synthetic and biotechnological derivative thereof, the method comprising:
introducing an expression vector into a host cell, the expression vector having an isolated nucleic acid molecule for encoding a monoclonal antibody, or an antigen-binding fragment thereof, or a synthetic and biotechnological derivative thereof that specifically binds kynurenine and acts as a detection marker for inflammation in an individual;
growing the cell under conditions permitting production of the monoclonal antibody or antigen-binding fragment thereof, or synthetic and biotechnological derivative thereof that specifically binds the kynurenine and acts as the detection marker for inflammation in the individual;
recovering the antibody or fragment or derivative so produced.

9. An in vitro method for detection of a disease or condition in an individual, wherein the level of kynurenine in a body fluid is determined using a monoclonal antibody, or an antigen-binding fragment thereof, or a synthetic and biotechnological derivative thereof that specifically binds kynurenine and acts as a detection marker for inflammation in the individual; and wherein the value of kynurenine measured in the individual to be diagnosed is compared with an average value obtained from a comparable cohort of healthy persons, whereby the value of kynurenine in the individual is increased.

10. The in vitro method according to claim 9, wherein the determination of kynurenine in the body fluid is used for therapy control in the individual,
and/or
monitoring of a health status of the individual.

11. The in vitro method according to claim 9, wherein the body fluid is serum, saliva and/or cerebro spinal fluid (CSF).

12. The in vitro method according to claim 9, wherein the level of kynurenine is at least two times higher in the individual than in the healthy control group.

13. The in vitro method according to claim 9, wherein the disease or condition is selected from at least one of neurodegenerative diseases, transplantation, infections, dialysis, dental diseases, sports medicine, gynecology, gastroentero-logy, cancer, diabetes, multiple sclerosis, asthma, and concussion.

14. The in vitro method according to claim 9, wherein the method is an ELISA test, or a lateral flow immunochromatographic assay, or a microfluidic test.

15. A test kit with which a level of kynurenine in a body fluid is determined using a monoclonal antibody, or an antigen-binding fragment thereof, or a synthetic and biotechnological derivative thereof that specifically binds kynurenine and acts as a detection marker for inflammation in an individual.
